(19) United States
(12) Patent Application Publication
(10) Pub. No.: US 2023/0372108 A1
(43) Pub. Date: Nov. 23, 2023

(54) GLENOID SYSTEM

(71) Applicant: Michael Greiwe, Edgwood, KY (US)

(72) Inventor: Michael Greiwe, Edgwood, KY (US)

(21) Appl. No.: 18/198,999

(22) Filed: May 18, 2023

Related U.S. Application Data
(60) Provisional application No. 63/344,243, filed on May 20, 2022.

(51) Int. Cl. A61F 2/30

(52) U.S. Cl. CPC A61F 2/30749; A61F 2002/4085

(57) ABSTRACT

A baseplate for implantation into a scapula includes a first circular portion, a second circular portion, and a third circular portion, wherein the first circular portion, the second circular portion, and the third circular portion define an upper surface a projection extending from the third circular portion in a direction away from an upper surface and configured to be received with an aperture created in the scapula; an aperture extending through the projection and configured to receive a bone screw such that a portion of the bone screw extends beyond an end of the projection and into the scapula, and wherein the aperture is configured to receive a projection of an artificial joint such that the artificial joint is coupled to the baseplate.

### CROSS-REFERENCE TO RELATED APPLICATION

This application claims the benefit of and priority to U.S. Provisional Patent Application No. 63/344,243, filed May 20, 2022, which is incorporated herein by reference in its entirety.

### FIELD

The present disclosure relates to apparatuses, systems, and methods for surgery. More particularly, the present disclosure relate to apparatuses, kits, systems, and methods of shoulder replacement.

### BACKGROUND

The human shoulder is susceptible to a number of injuries and ailments (e.g., osteoarthritis, rotator cuff injuries, shoulder dislocation, tumor of the shoulder joint, fractures, rheumatoid arthritis and other inflammatory disorders, osteonecrosis, etc.). In some situations, surgery may be recommended to address the injuries and ailments. For example, a patient may elect to have shoulder arthroplasty, otherwise referred to as total shoulder replacement surgery, or reverse total shoulder replacement. However, the hardware (e.g., implants) required for total shoulder replacement surgery and reverse total shoulder replacement surgery may be different.

In certain situations, some patients may have total shoulder replacement surgery and may also elect to subsequently have reverse total shoulder replacement surgery. For example, the total shoulder replacement surgery may be unsuccessful and/or the implanted hardware used during the total shoulder replacement surgery may wear out over time. Since total shoulder replacement surgery may involve removing damaged areas of bone, the overall bone structure of the shoulder may be weakened or otherwise compromised after the total shoulder replacement surgery. The weakened bone structure may lead to complications when performing a subsequent reverse total shoulder replacement surgery.

### SUMMARY

According to an aspect of the present disclosure, a base baseplate for implantation into a scapula is disclosed. The baseplate includes a first circular portion, a second circular portion, a third circular portion, wherein the first circular portion, the second circular portion, and the third circular portion define an upper surface, a projection extending from the third circular portion in a direction away from the upper surface and configured to be received with an aperture created in the scapula, and an aperture extending through the projection and configured to receive a bone screw such that a portion of the bone screw extends beyond an end of the projection and into the scapula, and wherein the aperture is configured to receive a projection of an artificial joint such that the artificial joint is coupled to the baseplate.

According to another aspect of the present disclosure, a surgical kit is disclosed. The surgical kit includes a baseplate configured to be implanted into an aperture created in a scapula, the baseplate including a first circular portion, a second circular portion, a third circular portion, wherein the first circular portion, the second circular portion, and the third circular portion define an upper surface, a projection extending from the third circular portion in a direction away from the upper surface and configured to be received with an aperture created in the scapula, an aperture extending through the projection, a bone screw configured to be inserted into the aperture in the baseplate such that a portion of the bone screw extends beyond an end of the projection and into the scapula, and an artificial joint defining an upper surface and including a projection extending away from the upper surface, wherein the projection is configured to be received within the aperture is configured such that the artificial joint is coupled to the baseplate.

According to another aspect of the present disclosure, a method of performing shoulder surgery is disclosed. The method includes removing a central portion of bone from a scapula to create a central aperture, removing a first portion of bone from the scapula to create a first aperture, removing a second portion of bone from the scapula to create a second aperture, inserting a projection of a baseplate into the central aperture, the baseplate further including a first circular portion received within the first aperture and a second circular portion received within the second aperture, and inserting a bone screw through an aperture extending through the projection of the baseplate, such that a portion of the bone screw extends past an end of the projection and into the scapula.

This summary is illustrative only and should not be regarded as limiting.

### BRIEF DESCRIPTION OF THE DRAWINGS

FIG. 1 is a perspective view of a glenoid system used in a total shoulder replacement surgery, according to an example embodiment.

FIG. 2 is a perspective view of a glenoid system used in a reverse total shoulder replacement, according to an example embodiment.

FIG. 3 is a side cross sectional view of a baseplate, according to an example embodiment.

FIG. 4 is a top view of the baseplate of FIG. 3.

FIG. 5A is a front view of a sizer, according to an example embodiment.

FIG. 5B is a side view of the sizer of FIG. 5A, according to an example embodiment.

FIG. 6A is a front view of a sizer, according to an example embodiment.

FIG. 6B is a side view of the sizer of FIG. 6A, according to an example embodiment.

FIG. 7A is a front view of a sizer, according to an example embodiment.

FIG. 7B is a side view of the sizer of FIG. 7A, according to an example embodiment.

FIG. 8A is a front view of a sizer, according to an example embodiment.

FIG. 8B is a side view of the sizer of FIG. 8A, according to an example embodiment.

FIG. 9 is a front view of the sizer of FIG. 5A with a guide wire inserted, according to an example embodiment.

FIG. 10 is a side view of a drill, according to an example embodiment.

FIG. 11A is a front view of a guide, according to an example embodiment.

FIG. 11B is a side view of the guide of FIG. 11A, according to an example embodiment.

FIG. 12A is a front view of a guide, according to an example embodiment.

FIG. 12B is a side view of the guide of FIG. 12A, according to an example embodiment.

FIG. 13A is a front view of a guide, according to an example embodiment.

FIG. 13B is a side view of the guide of FIG. 13A, according to an example embodiment.

FIG. 14A is a front view of a guide, according to an example embodiment.

FIG. 14B is a side view of the guide of FIG. 14A, according to an example embodiment.

FIG. 15 is a front view of the glenoid after the central aperture has been created, according to an example embodiment.

FIG. 16A is a top view of a reamer, according to an example embodiment.

FIG. 16B is a side view of the reamer of FIG. 16A.

FIG. 17 is a front view of the scapula, according to an example embodiment.

FIG. 18A is a top view of a guide, according to an example embodiment.

FIG. 18B is a side view of the guide of FIG. 18A, according to an example embodiment.

FIG. 19 is a side cross sectional view of a guide, according to an example embodiment.

FIG. 20 is a side view of a drill, according to an example embodiment.

FIG. 21 is a side view of a soft tissue protector, according to an example embodiment.

FIG. 22 is a side cross sectional view of a sphere coupled to a baseplate, according to an example embodiment.

FIG. 23 is a top view of an artificial joint, according to an example embodiment.

FIG. 24 is a side view of the artificial joint of FIG. 23.

FIG. 25 is a flow chart of a method of implanting a baseplate into the scapula, according to an example embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

The use of “e.g.” “etc.,” “for instance,” “in example,” and “or” and grammatically related terms indicates non-exclusive alternatives without limitation, unless otherwise noted. The use of “optionally” and grammatically related terms means that the subsequently described element, event, feature, or circumstance may or may not be present/occur, and that the description includes instances where said element, event, feature, or circumstance occurs and instances where it does not. The use of “attached” and “coupled” and grammatically related terms refers to the fixed, releasable, or integrated association of two or more elements and/or devices with or without one or more other elements in between. Thus, the term “attached” or “coupled” and grammatically related terms include releasably attaching or fixedly attaching two or more elements and/or devices in the presence or absence of one or more other elements in between. As used herein, the terms “proximal” and “distal” are used to describe opposing axial ends of the particular elements or features being described in relation to anatomical placement.

The human shoulder is generally referred to as a ball-and-socket joint that enables movement of the arm about the shoulder joint. A rounded head of the humerus bone interfaces with a shallow socket in the scapula, also referred to as the glenoid, to create the ball-and-socket joint. The shoulder joint may be susceptible to damage or injury that may limit the range of motion of the shoulder and/or cause pain during movement of the shoulder. For example, the shoulder joint may be susceptible to osteoarthritis, rotator cuff injuries, shoulder dislocation, tumor of the shoulder joint, fractures, rheumatoid arthritis and other inflammatory disorders, osteonecrosis, etc. In response to an injury or ailment, a person may undergo total replacement total shoulder replacement surgery, or shoulder arthroplasty, to improve the condition of his or her shoulder.

Total shoulder replacement surgery involves removing damaged areas of bone and replacing the bone with artificial implants. Generally, during total shoulder replacement, a portion of the rounded head of the humerus bone is replaced with a rounded spherical implant, which is also referred to as a glenosphere. The rounded spherical implant may be a polished metal ball with a stem attached. The stem is implanted into the humerus bone such that the polished metal ball extends from the humerus bone. Further, at least a portion of the glenoid is replaced with an artificial socket. For example, a groove or slot may be cut into the glenoid. The artificial socket may include a corresponding projection that is received by the slot in the glenoid. The artificial socket is then typically either press-fit or cemented into the glenoid. The artificial socket, which is typically manufactured from plastic, interfaces with the metal sphere to mimic the anatomy of a healthy shoulder joint to enable rotation of the arm about the shoulder joint.

In some situations, a person may undergo reverse total shoulder replacement surgery. For example, a total replacement surgery may fail or otherwise be unsuccessful (e.g., as a result of loosening of the artificial socket), and a reverse total shoulder replacement may be recommended. In this scenario, the artificial socket implanted into the glenoid during total shoulder replacement surgery may be removed from the scapula and a metal sphere may be implanted into the scapula. In reverse total shoulder replacement, the typical anatomy of the shoulder joint is reversed. In other words, the artificial socket is implanted into the humerus bone and the metal sphere is implanted into the glenoid of the scapula.

Performing a reverse total shoulder replacement surgery subsequent to a total shoulder replacement surgery may present a number of difficulties. For example, the bone structure of the scapula may be compromised as a result of total shoulder replacement, especially near the glenoid. For example, during total shoulder replacement surgery, one or more grooves or slots may be cut into the scapula to secure the artificial socket. These grooves or slots may reduce the structure strength of the scapula. Since the metal sphere is typically secured to the scapula via bone screws, compromised bone structure of the scapula may reduce the stability of the metal sphere implanted into the scapula.

Referring now to the figures generally, a glenoid system is disclosed according to various embodiments. The glenoid system may be modular such that the glenoid system can be used during both total shoulder replacement surgery and reverse total shoulder replacement surgery. According to various embodiments, the glenoid system includes a baseplate that may implanted into the scapula. The plate is configured to receive and couple to both a sphere (e.g., during a total shoulder replacement surgery) and an artificial socket (e.g., during a total shoulder replacement surgery). For example, the baseplate may be implanted during a total shoulder replacement surgery and an artificial socket may be coupled to the baseplate (e.g., via a Morse taper). According to various embodiments, the baseplate is secured to the scapula via one or more bone screws, thereby improving the stability of the baseplate and the artificial socket implanted during the total shoulder replacement surgery. As a result of the improved stability of the baseplate, the risk of glenoid loosening, or loosening of the artificial joint, may be reduced in total shoulder replacement surgery. Further, the baseplate may be an inlay baseplate, which may improve stability baseplate.

Due to the modular capabilities of the baseplate, performing a reverse total shoulder replacement surgery subsequent to a total shoulder replacement surgery may be simplified and the success rate of the total shoulder replacement surgery and reverse total shoulder replacement surgery may increase. For example, during the reverse total shoulder replacement surgery, rather than removing the artificial joint from the scapula, the artificial joint is removed from the baseplate, which does not alter the structural integrity of the scapula. The baseplate, which was previously implanted during the total shoulder replacement, is configured to receive a sphere during the reverse total shoulder replacement surgery. Since the baseplate can receive both an artificial socket and a sphere, there is no longer the need to cut additional grooves or drill additional bone screws into the scapula during the reverse total shoulder replacement surgery. Further, since the artificial socket is not coupled directly to the scapula during total shoulder replacement surgery, the bone structure will not be weakened or compromised as a result of removing the artificial socket.

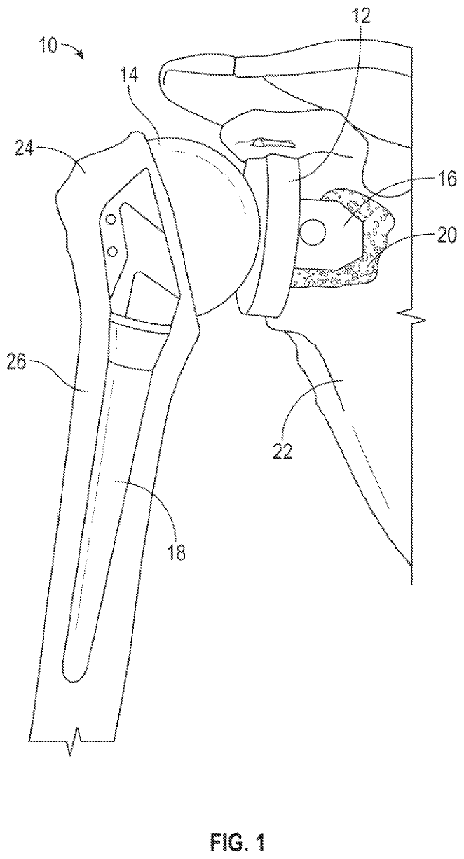

Referring now to FIG. 1, a glenoid system 10 used in a total shoulder replacement surgery is shown, according to an example embodiment. The glenoid system 10 includes an artificial joint 12 implanted into a scapula 22 and a metal sphere 14 implanted into a head 24 of a humerus 26. As shown, the artificial joint 12 includes a projection 16 that is implanted into an opening 20 in the scapula 22. For example, as a part of the total shoulder replacement surgery, the surgeon may remove a portion of the scapula 22 to create the opening 20. The projection 16 of the artificial joint 12 may then be press fit or cemented into the opening 20 to secure the artificial joint 12 to the scapula 22.

As shown, the metal sphere 14 is coupled to a stem 18. As a part of the total shoulder replacement surgery, a portion of the head 24 of the humerus 26 is removed. The stem 18 is then implanted (e.g., broached) into the head 24 of the humerus 26 such that the metal sphere 14 extends from the humerus 26 to mimic the portion of the head 24 that was removed. The metal sphere 14 then interfaces with the surface of the artificial joint 12 to enable rotation of the arm about the shoulder joint.

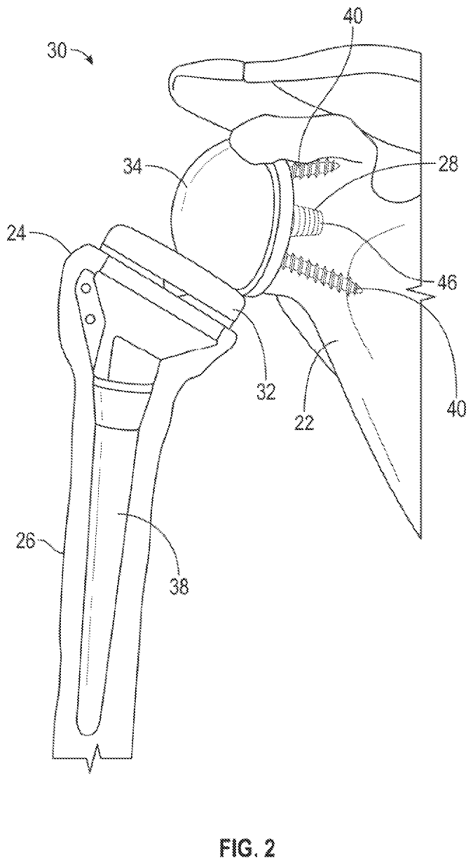

Referring now to FIG. 2, a glenoid system 30 used in a reverse total shoulder replacement surgery is shown, according to an example embodiment. The glenoid system 30 includes an artificial joint 32 implanted into the head 24 of the humerus 26 and a metal sphere 34 implanted into the scapula 22. As shown, the metal sphere 34 includes a projection 46 that is implanted into an opening 28 in the scapula 22. For example, as a part of the reverse total shoulder replacement surgery, the surgeon may remove a portion of the scapula 22 to create the opening 28. The projection 46 of the metal sphere 34 may then be press fit or cemented into the opening 28 to secure the metal sphere 34 to the scapula 22. Further, the metal sphere 34 is shown to be secured to the scapula via one or more bone screws 40.

As shown, the artificial joint 32 is coupled to a stem 38. As a part of the reverse total shoulder replacement surgery, a portion of the head 24 of the humerus 26 is removed. The stem 38 is then implanted (e.g., broached) into the head 24 of the humerus 26 such that the artificial joint 32 extends from the humerus 26 to mimic the portion of the head 24 that was removed. The artificial joint 32 then interfaces with the surface of the metal sphere 34 o enable rotation of the arm about the shoulder joint.

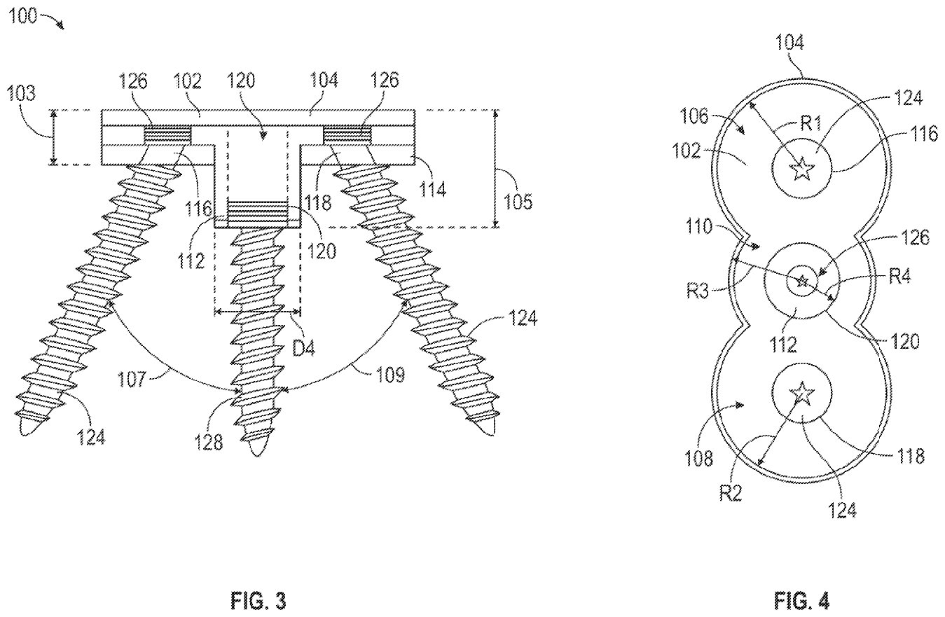

Referring now to FIGS. 3 and 4, a side cross sectional view and top view, respectively, of a baseplate 100 are shown according to an example embodiment. The baseplate 100 may be a part of a glenoid system and may be implanted into the scapula 22 proximate the glenoid. The baseplate 100 may be a modular baseplate 100 that is configured to receive both an artificial joint (e.g., as a part of a total shoulder replacement surgery) and a sphere (e.g., as a part of a reverse total shoulder replacement surgery). In this sense, the baseplate 100 may be used for both total shoulder replacement surgery and reverse total shoulder replacement surgery. Further, if the baseplate 100 is implanted into a patient as a part of total shoulder replacement surgery and the same baseplate 100 can be used for a subsequent reverse total shoulder replacement surgery, thereby simplifying the reverse total shoulder replacement surgery.

As shown, the baseplate 100 includes an upper surface 102 proximate the top of the baseplate 100. As shown, the upper surface 102 is substantially flat, however, according to other embodiments, the upper surface 102 may be curved (e.g., concave, convex, etc.). As shown, the baseplate 100 and the upper surface 102 form a “snowman” shaped body and define a first circular portion 106 that defines a first radius R1, a second circular portion 108 that defines a second radius R2, and a third circular portion 110 that defines a third radius R3. According to various embodiments, the snowman shaped body of the baseplate 100 may improve performance of the baseplate as the glenoid of the scapula 22 is generally oval shaped, rather that circular. According to various embodiments, R1, R2, and R3 are all substantially the same (e.g., within 5% of each other). For example, R1, R2, and R3 may each be between 5 mm and 40 mm. However, according to other embodiments, R1, R2, and R3 may not all be substantially the same. The size of the baseplate 100 and the size of R1, R2, and R3 may depend on the size of the patient's glenoid, as is discussed further herein. According to various embodiments, the upper surface 102 is manufactured from a biocompatible material. For example, the upper surface 102 may be manufactured from a plastic, such as polyethylene. According to other embodiments, the upper surface 102 may be manufactured from a metal, such as titanium, stainless steel, a mixed alloy, etc.

As shown, the baseplate 100 further includes an outer rim 104 extending outwardly from the upper surface 102. As shown, the outer rim 104 extends around the entire upper surface 102. According to various embodiments, the outer rim 104 extends between 0.5 mm and 3 mm past the upper surface 102. According to various embodiments, the baseplate 100 defines a height 103. According to various embodiments, the height 103 is between 5 mm and 10 mm. For example, the height 103 may be 7.5 mm. According to various embodiments, the outer rim 104 and/or some or all of the peripheral edge of the baseplate 100 may be manufactured from a porous material or may have a porous coating applied to it. According to various embodiments, the porous peripheral edge and/or outer rim 104 may improve stability of the baseplate 100 when implanted into the patient.

As shown, the baseplate 100 includes a projection 112 proximate the third circular portion 110 that extends from a lower surface 114 of the baseplate 100. According to various embodiments, the projection 112 is configured to be received by a central aperture, or an opening in the scapula, (e.g., as created by the surgeon using a drill or reamer) to secure the baseplate 100 to the scapula in an inlay manner. As shown, the distance between the upper surface 102 and the end of the projection 112 defines a depth 105 According to various embodiments, the depth 105 may be between 10 mm and 20 mm. For example, the depth 105 may be 15 mm. Further, the projection 112 defines a fourth radius R4 (i.e., a diameter D4). According to various embodiments, the fourth radius R4 is between 1.5 mm and 4.5 mm. For example, the fourth radius R4 may be 3 mm.

As shown, the baseplate 100 further defines a first opening 116 proximate the first circular portion 106, a second opening 118 proximate the second circular portion 108, and a third opening 120 proximate the third circular portion 110. For example, when viewed from above (e.g., FIG. 4), the first opening 116 may be at the center of the first circular portion 106, the second opening 118 may be at the center of the second circular portion 108, and the third opening 120 may be at the center of the third circular portion 110. As shown, the first opening 116 and the second opening 118 extend from the upper surface 102 through the lower surface 114 and the third opening 120 extends from the upper surface 102 through the projection 112. According to various embodiments, the first opening 116, the second opening 118, and the third opening 120 may define a radius between 3 mm and 9 mm. For example, the first opening 116, the second opening 118, and the third opening 120 may define a 5 mm radius.

As shown, the first opening 116, and second opening 118, and the third opening 120 are configured to individually receive a securing element (e.g., bone screw, bone barb, etc.). For example, the first opening 116 and the second opening 118 may be configured to individually receive a bone screw 124. According to various embodiments, the bone screws 124 may be utilized to secure the baseplate to the scapula. As shown, the bone screws 124 are configured to receive locking caps 126, which may reduce the likelihood of the bone screws 124 backing out of the baseplate 100 after the baseplate 100 is implanted. Further, as shown, the third opening 120 is configured to receive a locking screw 128 (e.g., a self-locking screw). By utilizing the locking screw 128, the likelihood of blackout of the locking screw 128 is reduced.

As shown, first opening 116 and the second opening 118 may be angled with respect to the upper surface 102 such that the bone screws 124 define a first angle 107 and a second angle 109, respectively, with respect to the locking screw 128. According to various embodiments, the first angle 107 and the second angle 109 may be the same or the first angle 107 and the second angle 109 may be different. According to various embodiments, the first angle 107 and the second angle 109 are between 0 degrees and 60 degrees.

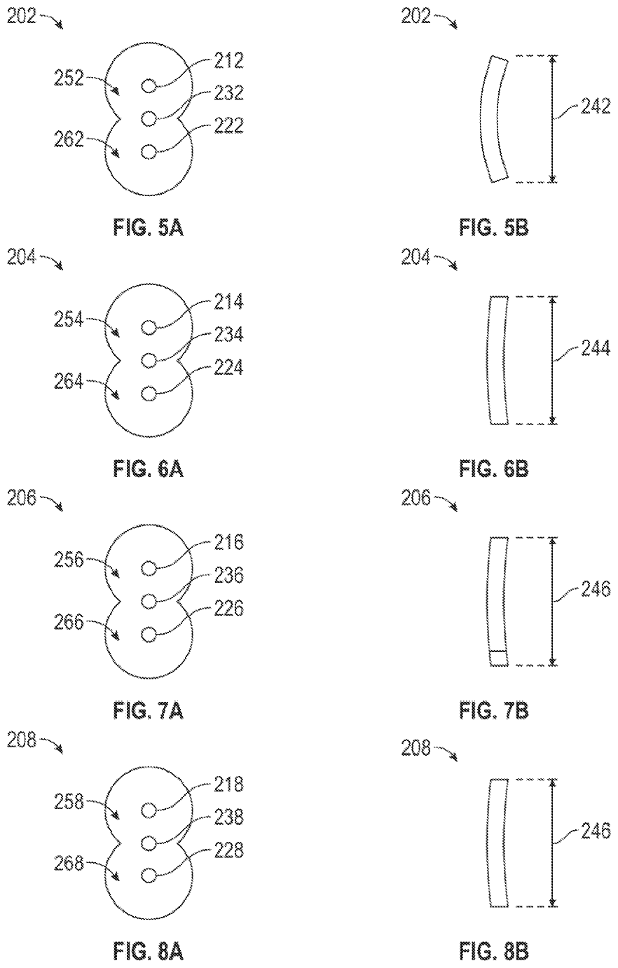

Referring now to FIGS. 5A-8B, a front view and a side view, respectively, of a plurality of sizers 202, 204, 206, and 208, are shown, according to an example embodiment. The plurality of sizers 202, 204, 206, and 208 may be used during implantation of the baseplate 100, as is discussed further herein. For example, after an incision is made and the surface of the glenoid is prepared, a surgeon may place each sizers 202, 204, 206, and 208 proximate the desired location of the baseplate to determine which size base plate best suits the particular patient's scapula anatomy.

Referring now to FIGS. 5A and 5B, a front view and a side view of a sizer 202 are shown, according to an example embodiment. As shown, the sizer 202 includes a first circular portion 252 and a second circular portion 262. The sizer 202 defines a length 242. For example, the length 242 may be 40 mm. Further, the sizer 202 includes a first opening 212 (e.g., proximate a center of the first circular portion 252), a second opening 222 (e.g., proximate a center of the second circular portion 262), and a third opening 232 positioned between the first opening 212 and the second opening 222. As is discussed further herein, the first opening 212, the second opening 222, and/or the third opening 232 may be configured to receive a guide pin and/or guide wire.

Referring now to FIGS. 6A and 6B, a front view and a side view of a sizer 204 are shown, according to an example embodiment. As shown, the sizer 204 includes a first circular portion 254 and a second circular portion 264. The sizer 204 defines a length 244. For example, the length 244 may be 44 mm. Further, the sizer 204 includes a first opening 214 (e.g., proximate a center of the first circular portion 254), a second opening 224 (e.g., proximate a center of the second circular portion 264), and a third opening 234 positioned between the first opening 214 and the second opening 224. As is discussed further herein, the first opening 214, the second opening 224, and/or the third opening 234 may be configured to receive a guide pin and/or guide wire.

Referring now to FIGS. 7A and 7B, a front view and a side view of a sizer 206 are shown, according to an example embodiment. As shown, the sizer 206 includes a first circular portion 256 and a second circular portion 266. The sizer 206 defines a length 246. For example, the length 246 may be 48 mm. Further, the sizer 206 includes a first opening 216 (e.g., proximate a center of the first circular portion 256), a second opening 226 (e.g., proximate a center of the second circular portion 266), and a third opening 236 positioned between the first opening 216 and the second opening 226. As is discussed further herein, the first opening 216, the second opening 226, and/or the third opening 236 may be configured to receive a guide pin and/or guide wire.

Referring now to FIGS. 8A and 8B, a front view and a side view of a sizer 208 are shown, according to an example embodiment. As shown, the sizer 208 includes a first circular portion 258 and a second circular portion 268. The sizer 208 defines a length 248. For example, the length 248 may be 54 mm. Further, the sizer 208 includes a first opening 218 (e.g., proximate a center of the first circular portion 258), a second opening 228 (e.g., proximate a center of the second circular portion 268), and a third opening 238 positioned between the first opening 218 and the second opening 228. As is discussed further herein, the first opening 218, the second opening 228, and/or the third opening 238 may be configured to receive a guide pin and/or guide wire.

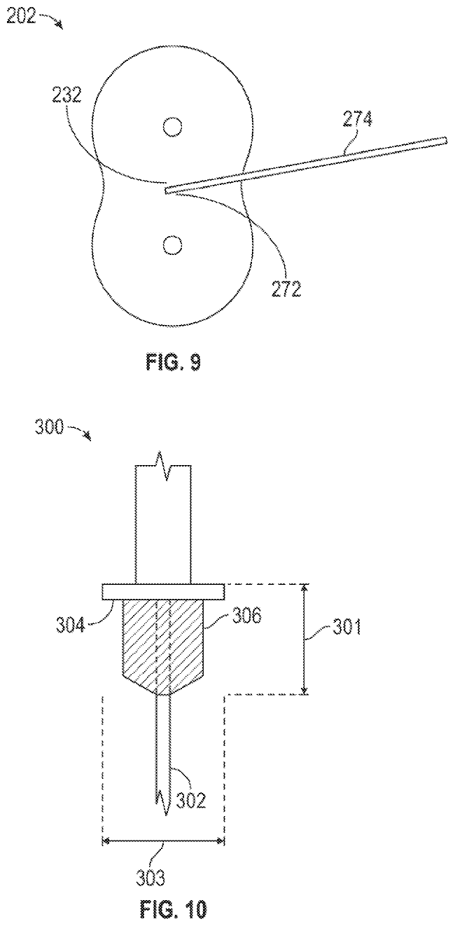

Referring now to FIG. 9, the sizer 202 is shown, according to an example embodiment. As shown, a pin 272 coupled to a guide wire 274 is inserted into the third opening 232 of the sizer 202. For example, as the sizer is held against the glenoid, the pin 272 may be inserted into the scapula of the patient. As is discussed below, the guide wire 274 may be used to guide a drill. It should be appreciated that while sizer 202 is shown, any of the other sizers discussed herein may be utilized in a similar manner.

Referring now to FIG. 10, a drill 300 is shown, according to an example embodiment. The drill 300 is configured to remove a portion of the scapula as a part of installation of the baseplate 100, as is discussed further herein. For example, the drill 300 may be used to drill a center aperture in the scapula. For example, the center aperture may be configured to receive the projection 112 of the baseplate 100. According to various embodiments, the drill 300 is a positive stop drill, as discussed below.

As shown, the drill 300 includes a guide member 302. The guide member 302 is configured to receive a guide wire (e.g., the guide wire 274) such that the drill 300 may rotate about the guide wire. The drill 300 may advance while the guide wire is positioned within the guide member 302 thereby enabling precision drilling of the center aperture, wherein the center of the center aperture is defined by the location that the pin 272 is inserted into the scapula. The guidewire further includes a drilling element 306. The drilling element 306 is configured to drill into a bone (e.g., the scapula). As shown, the drill element defines a width 303 such that the central aperture created by the drill 300 had a diameter equal to the width 303. According to various embodiments, the width 303 is between 2 mm and 10 mm. For example, the width 303 may be 5 mm, such that the central aperture has a diameter of 5 mm. Further, the drilling element 306 in combination with a stop 304 define a depth 301 (e.g., the vertical distance between the end of the drilling element 306 and the stop 304). For example, the drill 300 may create a central aperture that has a depth equal to the depth 301. According to various embodiments, the drill 300 will bottom out when the stop 304 contacts the scapula, thereby prevent the drill 300 from further drilling. According to various embodiments, the depth 301 is between 10 mm and 25 mm. For example, the depth 301 may be 15 mm. According to another example, the depth 301 may be 20 mm.

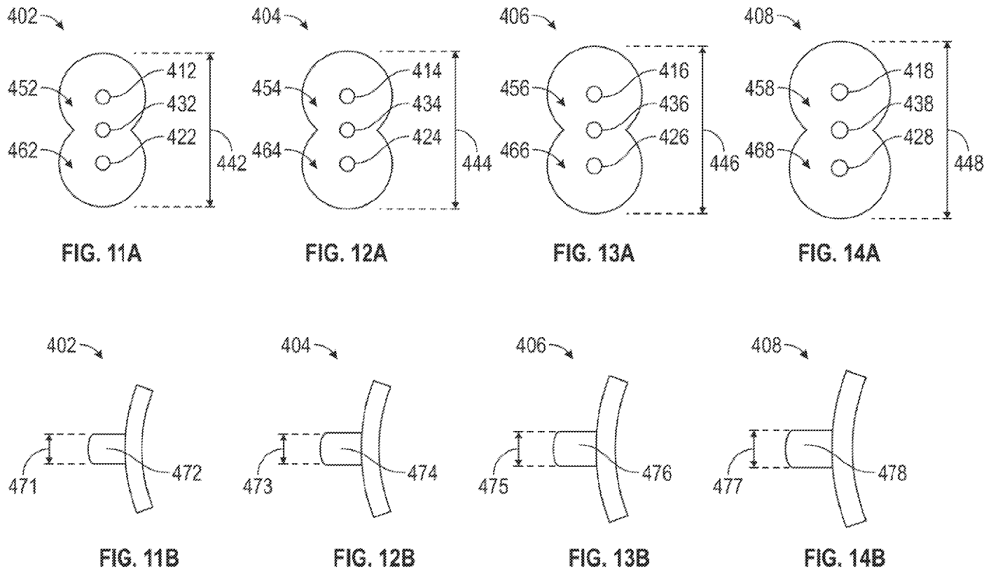

Referring now to FIGS. 11a-14b, a front view and a side view, respectively, of a plurality of guides 402, 404, 406, and 408, are shown, according to an example embodiment. The plurality of guides 402, 404, 406, and 408, may be used during implantation of the baseplate 100, as is discussed further herein. For example, after the central aperture is drilled (e.g., using drill 300), a surgeon may place one of the guides 402, 404, 406, and 408, over the central aperture. According to various embodiments, selecting one of the guides 402, 404, 406, and 408, may be based on the sizer that was previously selected and used. For example, if a 44 mm sizer was previously selected, a 44 mm guide may subsequently be selected.

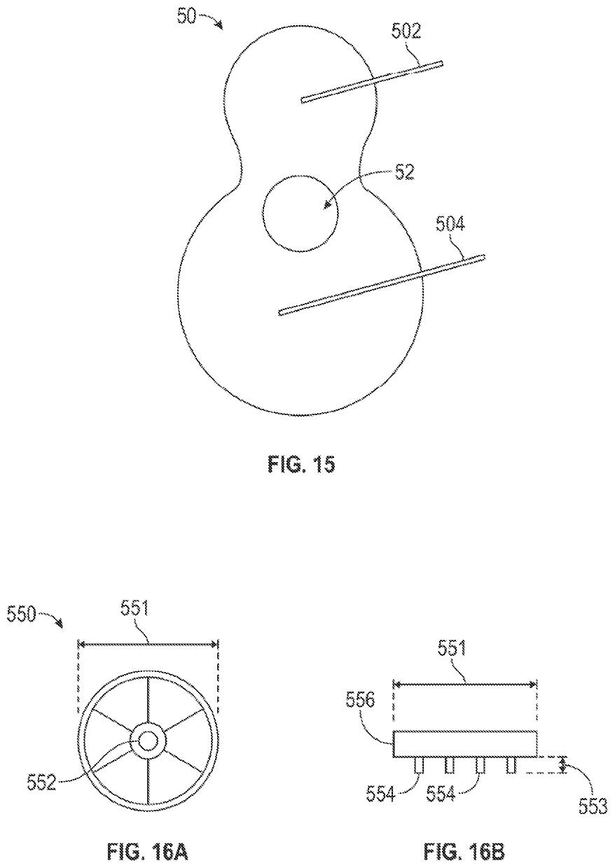

Referring now to FIGS. 11A and 11B, a front view and a side view of a guide 402 are shown, according to an example embodiment. As shown, the guide 402 includes a first circular portion 452 and a second circular portion 462. The guide 402 further defines a length 442. For example, the length 442 may be 40 mm. Further, the guide 402 includes a first opening 412 (e.g., proximate a center of the first circular portion 452), a second opening 422 (e.g., proximate a center of the second circular portion 462), and a third opening 432 positioned between the first opening 412 and the second opening 422. As is discussed further herein, the first opening 412, the second opening 422, and/or the third opening 432 may be configured to receive a pin (e.g., the pins 502 shown in FIG. 15).

As shown, the guide 402 further includes a projection 472 that defines a diameter 471. According to various embodiments, the projection 472 may be sized to fit within the central aperture that was drilled into the scapula (e.g., using the drill 300). For example, the diameter 471 may be equal to, or substantially equal to (e.g., within 1%, within 2%, within 3%, etc.) the width 303 of the drilling element 306 discussed above with respect to FIG. 10. In this example embodiment, the projection 472 may be inserted into the central aperture prior to the apertures being drilled though the first opening 412 and the second opening 422.

Referring now to FIGS. 12A and 12B, a front view and a side view of a guide 404 are shown, according to an example embodiment. As shown, the guide 404 includes a first circular portion 454 and a second circular portion 464. The guide 404 further defines a length 444. For example, the length 444 may be 44 mm. Further, the guide 404 includes a first opening 414 (e.g., proximate a center of the first circular portion 454), a second opening 424 (e.g., proximate a center of the second circular portion 464), and a third opening 434 positioned between the first opening 414 and the second opening 424. As is discussed further herein, the first opening 414, the second opening 424, and/or the third opening 434 may be configured to receive a pin (e.g., the pins 502 shown in FIG. 15).

As shown, the guide 404 further includes a projection 474 that defines a diameter 473. According to various embodiments, the projection 474 may be sized to fit within the central aperture that was drilled into the scapula (e.g., using the drill 300). For example, the diameter 471 may be equal to, or substantially equal to (e.g., within 1%, within 2%, within 3%, etc.) the width 303 of the drilling element 306 discussed above with respect to FIG. 10. In this example embodiment, the projection 474 may be inserted into the central aperture prior to the apertures being drilled though the first opening 414 and the second opening 424.

Referring now to FIGS. 13A and 13B, a front view and a side view of a guide 406 are shown, according to an example embodiment. As shown, the guide 406 includes a first circular portion 456 and a second circular portion 466. The guide 406 further defines a length 446. For example, the length 446 may be 48 mm. Further, the guide 406 includes a first opening 416 (e.g., proximate a center of the first circular portion 456), a second opening 426 (e.g., proximate a center of the second circular portion 466), and a third opening 436 positioned between the first opening 416 and the second opening 426. As is discussed further herein, the first opening 416, the second opening 426, and/or the third opening 436 may be configured to receive a pin (e.g., the pins 502 shown in FIG. 15).

As shown, the guide 406 further includes a projection 476 that defines a diameter 475. According to various embodiments, the projection 476 may be sized to fit within the central aperture that was drilled into the scapula (e.g., using the drill 300). For example, the diameter 471 may be equal to, or substantially equal to (e.g., within 1%, within 2%, within 3%, etc.) the width 303 of the drilling element 306 discussed above with respect to FIG. 10. In this example embodiment, the projection 476 may be inserted into the central aperture prior to the apertures being drilled though the first opening 416 and the second opening 426.

Referring now to FIGS. 14A and 14B, a front view and a side view of a guide 408 are shown, according to an example embodiment. As shown, the guide 408 includes a first circular portion 458 and a second circular portion 468. The guide 408 further defines a length 448. For example, the length 448 may be 54 mm. Further, the guide 408 includes a first opening 418 (e.g., proximate a center of the first circular portion 458), a second opening 428 (e.g., proximate a center of the second circular portion 468), and a third opening 438 positioned between the first opening 418 and the second opening 428. As is discussed further herein, the first opening 418, the second opening 428, and/or the third opening 438 may be configured to receive a pin (e.g., the pins 502 shown in FIG. 15).

As shown, the guide 408 further includes a projection 478 that defines a diameter 477. According to various embodiments, the projection 478 may be sized to fit within the central aperture that was drilled into the scapula (e.g., using the drill 300). For example, the diameter 471 may be equal to, or substantially equal to (e.g., within 1%, within 2%, within 3%, etc.), the width 303 of the drilling element 308 discussed above with respect to FIG. 10. In this example embodiment, the projection 478 may be inserted into the central aperture prior to the apertures being drilled though the first opening 418 and the second opening 428.

Referring now to FIG. 15, a front view of a glenoid 50 is shown, according to an example embodiment. According to various embodiments, FIG. 15 represent the glenoid after the central aperture 52 is drilled into the glenoid, a projection (e.g., the projection 472) of a guide (e.g., the guide 402) is placed into the central aperture 52, a first guide member (e.g., the guide pin 502, a guide wire, etc.) is inserted into a first opening (e.g., the first opening 512) a second guide member (e.g., the guide pin 502, a guide wire, etc.) is inserted into a second opening (e.g., the second opening 522), and the guide (e.g., the guide 402 is removed. The guide members are configured to be received by a drill or reamers (e.g., a positive stop reamer) and used as a center point to drill an aperture.

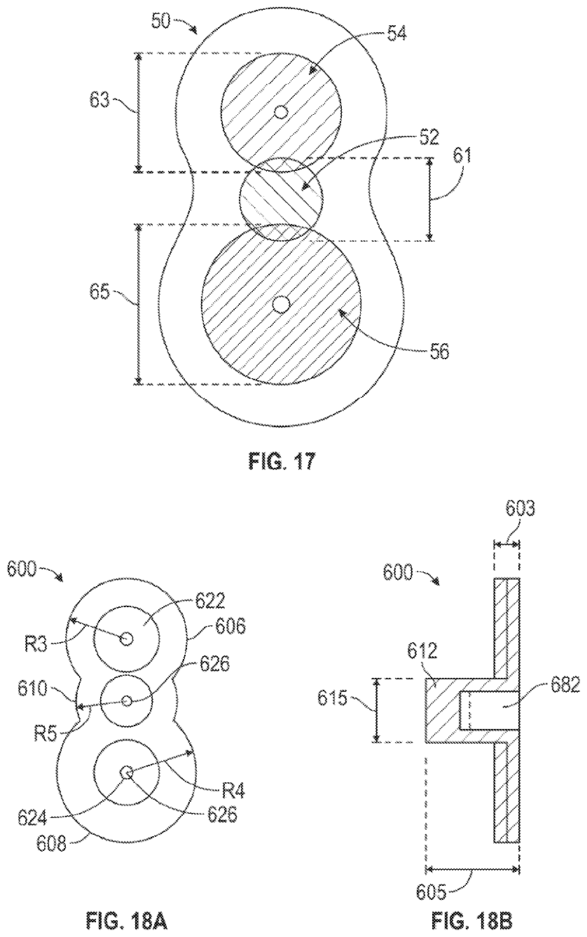

Referring now to FIGS. 16A and 16B, a top and side view of a reamer 550. The reamer 550 includes one or more cutters 554 that are configured to remove a portion of bone (e.g., from a scapula). The reamer defines a diameter 551 and a depth 553. As shown, the depth 553 is defined as the distance between a stop 556 and the end of the one or more cutters 554. As shown, the reamer 500 includes a center hole 552 that is configured to receive a guide member (e.g., the guide pin 502) such that the reamer 500 may rotate about the guide pin. For example, the reamer 550 may rotate about a first guide pin 502 to create a first aperture (e.g., the aperture 602 shown in FIG. 17) and about a second guide pin 502 to create a second aperture (e.g., the aperture 604 shown in FIG. 17).

According to various embodiments, the diameter 551 is between 15 mm and 30 mm. For example, according to various embodiments, the diameter 551 may be 20 mm, 22.5 mm, 24 mm, or 26 mm. According to various embodiments, the first aperture and the second aperture may be the same size (e.g., diameter and depth). However, according to other embodiments, a different reamer may be used to drill the first aperture and the second aperture such that the first aperture and the second aperture define a different diameter and/or depth.

Referring now to FIG. 17, a front view of the glenoid 50 is shown, according to an example embodiment. The glenoid 50 includes the central aperture 52 defining a central diameter 61, a first aperture 54 defining a first diameter 63, and a second aperture 56 defining a second diameter 65. According to various embodiments, the central diameter may be equal to or substantially equal to the width 303 and the first diameter 63 and the second diameter 65 may be equal to or substantially equal to the diameter 551.

Referring now to FIGS. 18A and 18B, a front and side view of a guide 600 are shown, according to an example embodiment. The guide 600 may be placed on top of the glenoid (e.g., as shown in FIG. 17) and used to drill guide holes, as is discussed further herein.

As shown, the guide 600 defines a first circular portion 606 that defines a first radius R3, a second circular portion 608 defining a second radius R4, and a third circular portion 110 defining a third radius R5. As shown, the guide 600 includes a projection 612 proximate the third circular portion 610 that extends from a lower surface 614 of the guide 600. According to various embodiments, the projection 612 is configured to be received by the central aperture 52 to at least partially secure the guide to the scapula. As shown, the guide 600 further defines a depth 605 corresponding with a distance the projection 612 extends from the remainder of the body of the guide 600. According to various embodiments, the depth 605 may be between 10 mm and 20 mm. For example, the depth 605 may be 15 mm. Further, the projection 612 defines a diameter 615. According to various embodiments, the diameter 615 is between 3 mm and 9 mm. For example, the diameter 615 may be 6 mm.

As shown, the guide 600 includes a first opening 622, a second opening 624 and a third opening 626. According to various embodiments, the first opening, the second opening, and/or the third opening 626 are configured to receive a drill. For example, guide holes may be drilled into the scapula through the guide 600 while the projection 612 is positioned within the central aperture 52. The guide holes may then be utilized to secure the baseplate 100 to the scapula via one or more bone screws, as is discussed further herein.

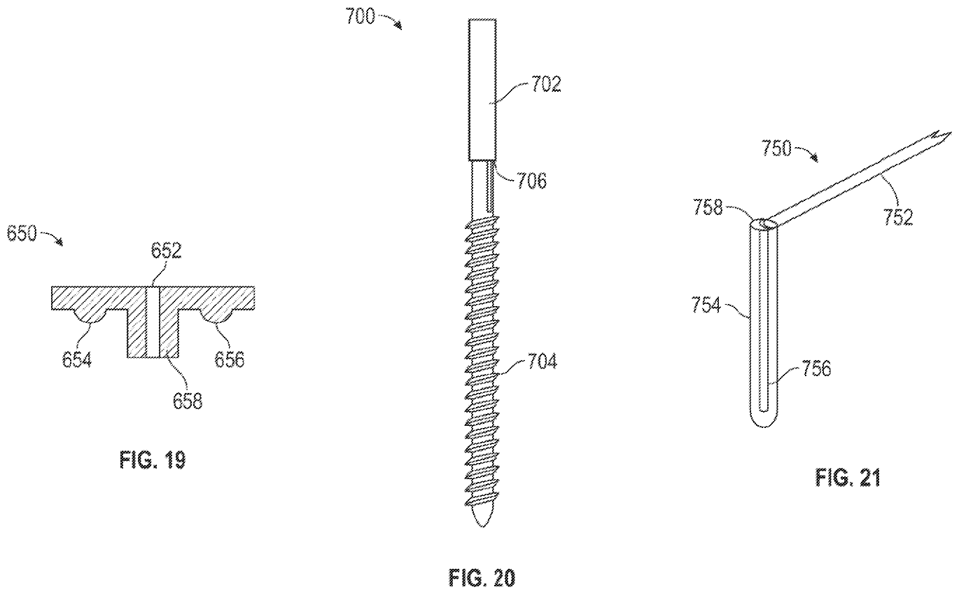

Referring now to FIG. 19, a guide 650 is shown, according to an example embodiment. The guide 650 may be used to drill a central guide hole in the scapula. The guide 650 includes a central opening 652 configured to receive a drill. For example, after the central aperture 52, the first aperture 54, and the second aperture 56 are created, a central projection 658 of the guide 650 may be placed into the central aperture 52. Further, a first projection 654 may be received by the first aperture 54 and a second projection 656 may be received within the second aperture 56. Once the guide 650 is in place, a central guide hole may be drilled in to the scapula through the central opening 652. The interaction between the first projection 654 and first aperture 54 and/or the interaction between the second projection 656 and the second aperture 56 may prevent movement of the guide 650 (e.g., rotation about the central projection 658) as the central guide hole is created in the scapula.

Referring now to FIGS. 20 and 21, a drill 700 and a soft tissue protector 750 are shown, according to an example embodiment. The drill 700 is configured to remove a portion of bone (e.g., the scapula). For example, the drill 700 may be used to drill a guide hole in the scapula, such as the central guide hole through the central opening 652 of the guide 650, a first guide hole though the first opening 622 of the guide 600, and/or a second guide hole through the second opening 624 of the guide 600.

As shown, the drill 700 includes a handle 702 such that a surgeon may hold the drill 700. Further, the drill 700 includes a cutting element 705 configured to remove a portion of bone. According to various embodiments, the cutting element 704 defines a diameter. According to various embodiments, the diameter is between 2 mm and 6 mm. According to an example embodiment, the diameter is 4.5 mm such that the drill 700 is configured to create a 4.5 mm guide hole (e.g., to accommodate a 5 mm bone screw).

According to various embodiments, the soft tissue protector 750 is configured to receive the cutting element 704 of the drill. For example, as shown, the soft tissue protector 750 includes an opening 756 in a body 754 of the soft tissue protector 750. The opening 756 is sized to receive the cutting element 705. A surgeon may utilize a handle 752 to hold the soft tissue protector 750 while drilling a guide hole with the drill 700 though the body 754 of the soft tide protector. As shown, the soft tissue protector 750 includes a shoulder 758 configured to interface with a stop 706 of the drill 700. For example, stop 706 may interface with the shoulder 758 while drilling the guide hole to prevent over-drilling and to create a guide hole of a desired depth.

After the guide holes are drilled, the baseplate 100 (see FIGS. 3 and 4) may be implanted into the scapula. For example, the projection 112 of the baseplate may be inserted into the central aperture 52. Further, the first circular portion 106 and the second circular portion 108 of the baseplate 100 may be received within the first aperture 54 and the second aperture 56 respectively. According to various embodiments, the baseplate 100 is inlay in the scapula. In other words, the baseplate 100 is submerged into the scapula as a result of the central aperture 52, the first aperture 54, and the second aperture 56. According to various embodiments, the baseplate 100 lay substantially flush within the scapula. Once the baseplate 100 is in a desired position, the baseplate 100 may be secured using one or more bone screws (e.g., the bone screws 124, the locking screw 128, etc.), as shown in FIG. 3.

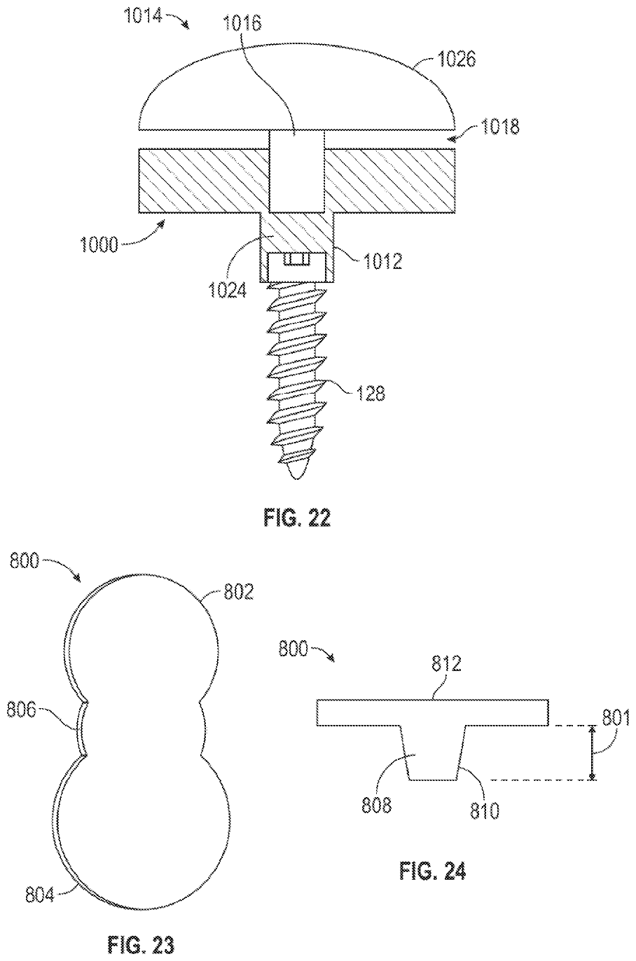

Referring now to FIG. 22, a side cross sectional view of a sphere 1014 coupled to a baseplate 1000 is shown, according to an example embodiment. For example, the sphere 1014 may be coupled to the baseplate 1000 as a part of a reverse total shoulder replacement surgery. According to various embodiments, the baseplate 1000 may be similar to the baseplate 100 discussed herein. For example, the baseplate 1000 may be identical to the baseplate 100. As shown, the baseplate is coupled to the scapula using a locking screw 128. It should be appreciated that while the baseplate 1000 is shown without the bone screws 124 installed, according to an example embodiment. However, it should be appreciated that, according to other embodiments, the baseplate 1000 may utilize one or more bone screws 124 to secure the baseplate 1000 to the scapula.

As shown, the baseplate 1000 includes a projection 1012 extending from the remainder of the baseplate. The projection includes an aperture 1024. The aperture 1024 is configured to receive a projection 1016 of the sphere 1014 to couple the sphere 1014 to the baseplate 1000. According to various embodiments, the aperture 1024 includes a Morse taper and the projection 1016 includes a counterpart Morse taper to secure the projection 1016 within the aperture 1024. According to various embodiments, the projection 1016 may be pressure fit into the aperture 1024. For example, the projection 1016 and/or the aperture 1024 may deform (e.g., elastically or plastically) when the projection 1016 is inserted into the aperture 1024 to secure the sphere 1014 to the baseplate 1000.

According to various embodiments, a gap 1018 exists between the sphere 1014 and the baseplate 1000. According to various embodiments, the projection 1016 may bottom out or otherwise be prevented from past a predetermined point within the aperture 1024 such that the gap 1018 is created. According to various embodiments, the gap 1018 may ease the difficulty of extraction of the sphere 1014 from the baseplate 1000. According to various embodiments, the gap 1018 may be between 1 mm and 6 mm. For example, the gap 1018 may be between 3 mm and 4 mm.

According to various embodiments, the baseplate 1000 may be manufactured from a biocompatible metal or a biocompatible plastic, such as polyethylene, or any combination thereof. Further, the sphere 1014 may be manufactured from a biocompatible metal or a biocompatible plastic, such as polyethylene, or any combination thereof. According to various embodiments, the projection 1016 is manufactured from the same material as a glenosphere portion 1026 (e.g., from a biocompatible metal). However, according to various embodiments, the projection 1016 is manufactured from a different material than the glenosphere portion 1026.

Referring now to FIGS. 23 and 24, top and side view of an artificial joint 800 are shown, according to an example embodiment. The artificial joint 800 is configured to be coupled to a baseplate (e.g., the baseplate 100, the baseplate 1000, etc.), as a part of a total shoulder replacement surgery. For example, as shown, the artificial joint 800 includes projection 808 that is configured to be received within an aperture (e.g., the third opening 120, the aperture 1024, etc.). The projection defines a depth 801. According to various embodiments, the depth 801 is between 5 mm and 9 mm. For example, the depth 801 may be 7 mm. According to various embodiments, the projection 808 includes a Morse taper 810 that is configured to interface with a counterpart Morse taper of the aperture to couple the artificial joint 800 to the baseplate. According to various embodiments, the projection 808 may be pressure fit into the aperture. For example, the projection 808 and/or the aperture may deform (e.g., elastically or plastically) when the projection 808 is inserted into the aperture to secure the artificial joint 800 to the baseplate. According to various embodiments, the baseplate 1000 may be manufactured from a biocompatible metal or a biocompatible plastic, such as polyethylene, or any combination thereof.

According to various embodiments, a gap exists between the artificial joint 800 and the baseplate after the baseplate is installed. According to various embodiments, the projection 1016 may bottom out or otherwise be prevented from past a predetermined point within the aperture such that the gap is created. According to various embodiments, the gap may ease the difficulty of extraction of the artificial joint 800 from the baseplate (e.g., as a part of converting the total shoulder replacement to a reverse total shoulder replacement). According to various embodiments, the gap may be between 1 mm and 6 mm. For example, the gap may be between 3 mm and 4 mm.

As shown, the artificial joint 800 includes a first circular portion 802, a second circular portion 804 and a third circular portion 806, which may align with the circular portions of the baseplate. Further, the artificial joint 800 defines an upper surface 812. According to various embodiments, the upper surface 812 is flat. However, according to various embodiments, the upper surface 812 may include a curvature (e.g., concave) to mimic the natural curvature of the glenoid.

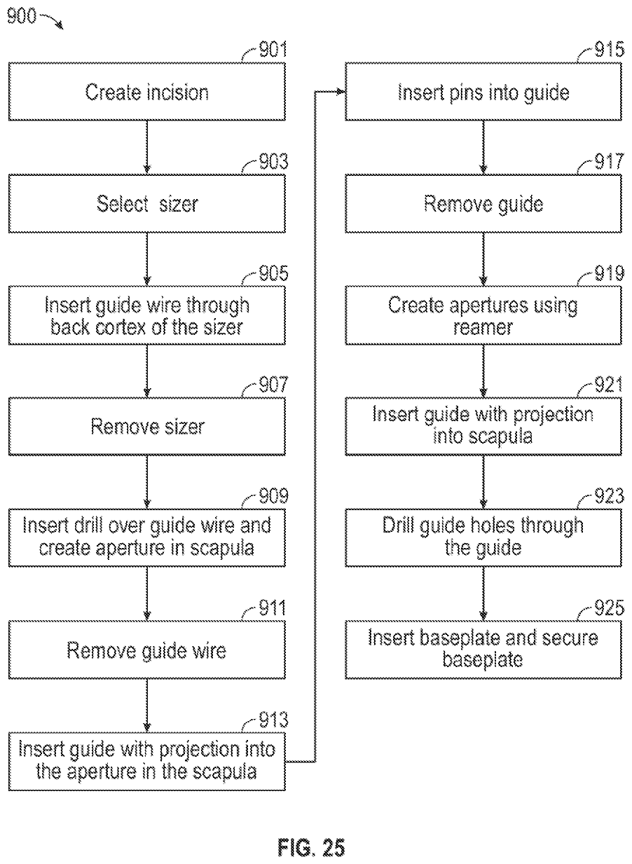

Referring now to FIG. 25, a method of installing a baseplate into a scapula 900 is shown according to an example embodiment. As discussed further herein, the baseplate may be configured to receive both an artificial joint (e.g., as a part of a total shoulder replacement) and a sphere (e.g., as a part of a reverse total shoulder replacement). According to various embodiments, the baseplate may be installed as a part of a total shoulder replacement surgery, coupled to an artificial joint, de-coupled decoupled from the artificial joint (e.g., as a part of a conversion from a total shoulder replacement and a reverse total shoulder replacement), and couple to a sphere (e.g., as a part of a reverse total shoulder replacement). Since the baseplate is usable with both an artificial joint and sphere, additional modifications to the scapula need not be made during a conversion from a total shoulder replacement to a reverse total shoulder replacement, thereby reducing the complexity of the conversion surgery and maintaining structural integrity of the scapula during the conversion.

The method 900 may be used to install any of the baseplates (e.g., baseplate 100, baseplate 1000, etc.) described herein. It should be appreciated that the method need not be performed in the order shown. Further, various processes may be omitted and additional processes may be added to the method 900.

At process 901, an incision is made. For example, and incision may be made proximate the shoulder of a patient to enable access to the scapula and the glenoid. According to various embodiments, process 901 may include removing a portion of bone from the scapula to prepare the glenoid for either a total shoulder replacement or a reverse total shoulder replacement.

At process 903, a sizer is selected. For example, one of the sizers 202, 204, 206, 208 may be selected. Process 903 may involve holding the various sizers proximate the glenoid. Once the surgeon determines the best for the patient, that sizer is selected. It should be appreciated that the size of the guides and baseplate that are selected later during the method 900 may be predetermined based on the sizer selected at process 903. For example, if the sizer 204 is selected, the guide 404 may be pre-selected and used later during the method 900.

At process 905, a guide wire is inserted through the back cortex of the sizer. For example, the guide wire 274 may be coupled to a pin 272 that is inserted through an opening (e.g., the third opening 232, 234, 236, 238) in a central portion of the guide and into the scapula such that the pin 272 is coupled to the scapula and the guide wire extends away from the scapula, as shown in FIG. 9 above. At process 907, the sizer is removed while the guide wire remains attached to the scapula.

At process 909, a drill is inserted over the guide wire to and is used to create an aperture in the scapula. For example, the drill 300 may be used to create the central aperture 52 in the scapula. According to various embodiments, the pin inserted into the scapula defines the center of the aperture. At process 911, the guide wire are removed from the scapula. For example, the pin 272 may be removed from the scapula to decouple the guide wire 274 from the scapula.

At process 913, a guide with a projection is inserted into the aperture of the scapula. For example, the guide 402 having projection 472 may be inserted into the central aperture 52 of the scapula. Alternatively, any of the other guides discussed herein may be inserted into the aperture (e.g., the guide 404, the guide 406, the guide 408, etc.).

At process 915, pins are inserted into the guide and into the scapula. For example, the first guide pin 502 may be inserted into the scapula through the first opening 412 and/or the second pin 504 may be inserted into the scapula though the second opening 422 of the guide 402. At process 917, the guide is removed from the scapula. For example, the guide 402 may be removed from the scapula. After removing the guide, the one or more pins will remain positioned within the scapula (e.g., as shown in FIG. 15).

At process 919, a first aperture and a second aperture are created using a reamer. For example, the first aperture 54 and the second aperture 56 may be created using the reamer 550. The reamer may rotate about the pins such that the first aperture and the second aperture are centered about the pins. After the first aperture and the second aperture are created, the pins may be removed from the scapula.

At process 921, a guide with a projection is inserted into the scapula. For example, the guide 600 may be inserted into the scapula such that the projection 612 is received within the central aperture 52. At process 923, guide holes are drilled into the scapula thought the guide. For example, the drill 700 may be inserted through the opening 756 in the soft tissue protector 750 and through the first opening 622 and/or the second opening 624 to create the guide holes in the scapula. According to various embodiments, a central guide hole may be drilled through the third opening 626. However, according to various other embodiments, the guide 600 may be removed, and an alternative guide 650 may be inserted such that the central guide hole may be drilled thought the central opening 652 of the guide 650.

At process 925, the baseplate is inserted into the scapula and secured to the scapula. For example, according to various embodiments, the bone screws 124 may be drilled into the respective guide hole through the first opening 116 and the second opening 118. The locking screw 128 may then subsequently be drilled into the respective guide hole through the third opening 120 of the baseplate 100. Once the baseplate 100 is secured, locking caps 126 may be installed onto the ends of the bone screws 124, which may reduce the risk of back out of the bone screws 124.

After the baseplate is installed into the scapula, an artificial joint may be coupled to the baseplate as a part of a total shoulder replacement surgery. Alternatively, a sphere may be coupled to the baseplate as a part of a reverse total shoulder replacement surgery.

As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. The devices, systems and methods described herein may be embodied in other specific forms without departing from the characteristics thereof. The foregoing implementations are illustrative rather than limiting of the described systems and methods. The scope of the systems and methods described herein is thus indicated by the appended claims, rather than the foregoing description, and changes that come within the meaning and range of equivalency of the claims are embraced therein.

What is claimed is:

1. A baseplate for implantation into a scapula, the baseplate comprising:
a first circular portion;
a second circular portion;
a third circular portion, wherein the first circular portion, the second circular portion, and the third circular portion define an upper surface;
a projection extending from the third circular portion in a direction away from the upper surface and configured to be received with a scapula aperture created in the scapula; and
an aperture extending through the projection and configured to receive a bone screw such that a portion of the bone screw extends beyond an end of the projection and into the scapula, and wherein the aperture is configured to receive a joint projection of an artificial joint such that the artificial joint is coupled to the baseplate.

2. The baseplate of claim 1, wherein the aperture extending through the projection is further configured to receive a sphere projection extending from a sphere such that the sphere is coupled to the baseplate.

3. The baseplate of claim 1, wherein the aperture extending through the projection includes a Morse taper.

4. The baseplate of claim 1, wherein the aperture is a central aperture, the bone screw is a central bone screw, and the baseplate further comprises a first aperture extending through the first circular portion and a second aperture extending through the second circular portion, the first aperture being configured to receive a first bone screw and the second aperture being configured to receive a second bone screw.

5. The baseplate of claim 4, wherein the first aperture is angled away from the central aperture such that the first bone screw is not parallel to the central bone screw and the second aperture is angled away from the central aperture such that the second bone screw is not parallel to the central bone screw.

6. The baseplate of claim 1, further comprising an outer ring surrounding an outer edge of the upper surface of the baseplate.

7. The baseplate of claim 1, wherein the aperture is configured to prevent the joint projection of the artificial joint from extending beyond a predetermined point within the aperture such that a gap exists between the upper surface and the artificial joint.

8. A surgical kit, comprising:
a baseplate configured to be implanted into a scapula aperture created in a scapula, the baseplate comprising:
a first circular portion;
a second circular portion;
a third circular portion, wherein the first circular portion, the second circular portion, and the third circular portion define an upper surface;
a projection extending from the third circular portion in a direction away from the upper surface and configured to be received within the scapula aperture created in the scapula;
an aperture extending through the projection;
a bone screw configured to be inserted into the aperture in the baseplate such that a portion of the bone screw extends beyond an end of the projection and into the scapula; and
an artificial joint defining an upper joint surface and including a joint projection extending away from the upper joint surface, wherein the joint projection is configured to be received within the aperture is configured such that the artificial joint is coupled to the baseplate.

9. The surgical kit of claim 8, further comprising a sphere having a sphere projection, the projection is configured to be received within the aperture such that the artificial joint is coupled to the baseplate.

10. The surgical kit of claim 8, wherein the aperture in the baseplate extending through the projection includes a Morse taper and the projection of the artificial joint includes a corresponding Morse taper.

11. The surgical kit of claim 8, wherein the aperture in the baseplate is a central aperture, the bone screw is a central bone screw, and the baseplate further comprises a first aperture extending through the first circular portion and a second aperture extending through the second circular portion, the first aperture being configured to receive a first bone screw and the second aperture being configured to receive a second bone screw.

12. The surgical kit of claim 11, wherein the central bone screw is a locking screw and the first bone screw and the second bone screw are configured to receive a locking cap.

13. The surgical kit of claim 8, wherein the baseplate further comprises an outer ring surrounding an outer edge of the upper surface of the baseplate.

14. The surgical kit of claim 8, wherein the aperture extending through the projection of the baseplate is configured to prevent the projection of the artificial joint from extending beyond a predetermined point within the aperture such that a gap exists between the upper surface and the artificial joint.

15. A method of performing shoulder surgery, the method comprising:
removing a central portion of bone from a scapula to create a central scapula aperture;
removing a first portion of bone from the scapula to create a first scapula aperture;
removing a second portion of bone from the scapula to create a second scapula aperture;
inserting a projection of a baseplate into the central scapula aperture, the baseplate further comprising a first circular portion received within the first scapula aperture and a second circular portion received within the second scapula aperture; and
inserting a bone screw through an aperture extending through the projection of the baseplate, such that a portion of the bone screw extends past an end of the projection and into the scapula.

16. The method of claim 15, further comprising inserting a joint projection of an artificial joint into the aperture extending through the projection such that the artificial joint is coupled to the baseplate.

17. The method of claim 15, further comprising inserting a sphere projection of a sphere into the aperture extending through the projection such that the sphere is coupled to the baseplate.

18. The method of claim 15, wherein removing the central portion of bone from the scapula to create the central scapula aperture includes:
placing one or more sizers proximate the scapula;
selecting an appropriate sizer from the one or more sizers;
inserting a guide wire through a central aperture of the appropriate sizer;
removing the appropriate sizer from the scapula; and
inserting a drill over the guide wire to create the central scapula aperture.

19. The method of claim 18, wherein removing the first portion of bone and the second portion of bone includes:
selecting a guide;
inserting a guide projection of the guide into the central scapula aperture;
placing a first pin into a first aperture of the guide and a second pin into a second aperture of the guide;
remove the guide from the scapula;
insert a reamer over the first pin and create the first scapula aperture; and
insert the reamer over the second pin and create the second scapula aperture.

20. The method of claim 19, wherein the guide is selected from a plurality of guides based on the appropriate sizer that was selected.
